(19) United States
(12) Patent Application Publication
(10) Pub. No.: US 2022/0055780 A1
(43) Pub. Date: Feb. 24, 2022

(54) LIDDING FILM FOR ASEPTIC PACKAGING

(71) Applicant: Sonoco Development, Inc., Hartsville, SC (US)

(72) Inventors: JOSEPH D. GAGNE, Holyoke, MA (US); Randy Koteles, Hartsville, SC (US); Dominic Savino, Hartsville, SC (US); Kristen Kenlin, Hartsville, SC (US)

(21) Appl. No.: 17/408,017

(22) Filed: Aug. 20, 2021

Related U.S. Application Data
(60) Provisional application No. 63/068,451, filed on Aug. 21, 2020.

(51) Int. Cl. B65B 7/28; B65B 55/02; B32B 27/32; B32B 27/08; B32B 37/16

(52) U.S. Cl. CPC B65B 7/2878; B65B 55/02; B32B 27/32; B32B 27/08; B32B 2323/10; B32B 2255/10; B32B 2307/514; B32B 2323/043; B32B 2435/02; B32B 37/16

(57) ABSTRACT

A method for providing a film lidding structure which can survive aseptic processing, the method comprising: providing a high-density polyethylene (HDPE) or polypropylene (PP) film layer; laminating the HDPE or PP film layer to at least one base layer to form a lidding; adhering the at least one base layer to the rim of a container to form a lidded container; and sterilizing the lidded container using high concentration hydrogen peroxide at elevated temperature.

### CROSS-REFERENCE TO RELATED APPLICATIONS

This application claims priority to U.S. Provisional Patent Application No. 63/068,451, filed Aug. 21, 2020, entitled “LIDDING FILM FOR ASEPTIC PACKAGING”, wherein the foregoing is incorporated by reference in its entirety herein.

### FIELD OF THE INVENTION

The present invention relates generally to a film lidding structure for aseptic packaging which can be sterilized under high concentration hydrogen peroxide and heat.

### BACKGROUND

Typical lidding has historically used oriented polyethylene terephthalate (PET) film (O-PET or OPET) as the exterior surface film due to its heat resistance, which is critical during the sealing process. In many cases, the OPET is reverse printed to provide the lidding with graphics, instructions, logos, or the like. However, if a reverse printed OPET film is used as the outer layer in a laminated structure and that lidding goes through a sterilization process that utilizes hydrogen peroxide at a high concentration (˜35%) at elevated temperatures, the lidding may internally delaminate or fail. OPET does not have the chemical resistance to survive such conditions. Such aseptic processing may also cause a smearing of ink and/or softening of adhesive, resulting in distortion and delamination, or other alteration of the printed material, as the printed ink is no longer securely sealed within the laminate. The present invention provides a reverse-printed lidding material which can survive a 35% hydrogen peroxide sterilization process (or greater concentrations) at an elevated temperature.

### SUMMARY OF THE INVENTION

The inventors have found that high-density polyethylene (HDPE) or polypropylene (PP) film has a superior chemical resistance to high concentration hydrogen peroxide solutions at elevated temperatures. Though not wishing to be bound to any theory, HDPE and/or PP film is believed to prevent the migration of the peroxide through the film, avoiding the noted negative impacts on the inks and adhesives used in the lamination or formation process. The HDPE and/or PP films used in the present reverse-printed laminated structure may survive far longer than the currently used PET or OPET structures, both during the sterilization process and thereafter. The present invention may improve the efficiency of the sterilization process by allowing the sterilization machinery to idle during the sterilization process without concerns that the lidding materials will be negatively impacted.

### DETAILED DESCRIPTION

Reference will now be made in detail to embodiments of the invention. Each example is provided by way of explanation of the invention, not limitation of the invention. In fact, it will be apparent to those skilled in the art that modifications and variations can be made in the present invention without departing from the scope or spirit thereof. For instance, features illustrated or described as part of one embodiment may be used on another embodiment to yield a still further embodiment. Thus, it is intended that the present invention covers such modifications and variations as come within the scope of the appended claims and their equivalents.

In an embodiment, the invention comprises a multilayer lidding film. In other embodiments, the invention may comprise a flexible film material which can be formed into a package, such as a pouch, bag, stand-up pouch or the like. The lidding film or packaging material may be useful for sealing containers or forming packages, as the case may be, which contain food products such as fresh fruit and produce, dairy products such as yogurt, sour cream, or the like, pharmaceuticals, personal care products, chemical products, cleaning products, or any other products known in the art. In an embodiment, the lidding film or packaging material is useful for containers or packages which are aseptic. “Aseptic,” as used herein, refers to a process wherein a container or packaging material is sterilized in a hygienic environment. “Aseptic” is also used herein to refer to the resulting sterilized container or package. The container, package, or packaging material may be sterilized before filling with a product or may be sterilized after filling and sealing, in certain embodiments. If the aseptic container or package contains a food product, the food product may be rendered shelf-stable in normal nonrefrigerated conditions. In an embodiment, the food product may be processed under ultra-high temperature pasteurization processes.

In a particular embodiment, the film may be useful as a lidding material for sealing containers that comprise a molded or shaped container, a spirally-wound container, or any other container defining a cavity. The container of the invention may have a bottom wall and one or more sidewalls extending upwardly from the base or bottom wall. The container bottom and at least one sidewall may define the internal cavity of the container. The one or more sidewalls may terminate in an edge, rim, or flange. In an embodiment, the lidding film of the invention is adhered to the edge, rim, or flange of the container. In an embodiment, the lidding film may be removably adhered, resealably adhered, or permanently adhered to the container edge, rim or flange. If a flange is present, the film lidding may be applied to the top surface of the flange.

In an embodiment, the film lidding comprises a multilayer structure. In a particular embodiment, the first or outermost layer of the film structure may comprise HDPE or PP. In an embodiment, the HDPE or PP layer is reverse printed, such as via a reverse printing station. To improve the adhesion of the ink to the inside surface of the HDPE or PP layer, the relevant surface can be treated or modified before printing. Suitable surface treatments and modifications can include mechanical treatments (such as corona treatment, plasma treatment, and flame treatment) and/or primer treatment. Surface treatments and modifications are known to those of skill in the art. The ink system should be capable of withstanding, without diminished performance, the temperature ranges to which the trap printed image will be exposed during lamination, sterilization, heat sealing (during pouch production), packaging (pouch filling), distribution, and the like.

In an embodiment, one or more polyethylene-based film layers may additionally be present in the multilayer film structure. As used herein, the term “polyethylene-based” or “PE-based” may include high density polyethylene (HDPE), low density polyethylene (LDPE), medium density polyethylene (MDPE), ultra high molecular weight polyethylene (UHMWPE), cross-linked polyethylene (PEX), linear low density polyethylene (LLDPE), very low density polyethylene (VLDPE), polyethylene copolymers, chemically-modified polyethylene, and/or any other PE-based film material. Any polyethylene material known in the art may be utilized in the invention. In an embodiment, a PE-based material comprises any material with the chemical formula (C₂H₄)ₙ.

In an embodiment, one or more paper-based layers and/or barrier layers may additionally be present in the multilayer film structure. In an embodiment, one or more metal or metalized layers may be present within the film structure. In an embodiment, a foil layer may be present within the film structure.

The various film layers may be adhered together using any adhesive or methods known in the art. In an embodiment, the adhesive may comprise high temperature resistant adhesives. In an embodiment, one or more of the layers may be co-extruded with an adhesive. In other embodiments, an adhesive layer may be separately applied to the underside of the outer, reverse-printed HDPE or PP layer, such as at an adhesive station before being adhered to another layer.

Additional materials that can be incorporated into the lidding film may include antiblock agents, slip agents, antifog agents, fillers, pigments, dyestuffs, antioxidants, stabilizers, processing aids, plasticizers, fire retardants, UV absorbers, etc.

In a particular embodiment, the layers may comprise, from outer to inner, HDPE/ink/adhesive/foil/adhesive/cast polypropylene. In this embodiment, the HDPE or PP layer may comprise the outermost layer and the cast polypropylene may comprise the innermost layer (relative to the interior of the container to which the lidding is applied.

In an embodiment, the HDPE or PP layer may be printed on its outer surface and an overlacquer may be applied to the exterior of the ink/HDPE layer or ink/PP layer. In an embodiment, the overlacquer may be heat-resistant and/or may provide heat resistance to the film lidding. In an embodiment, the overlacquer may be electron-beam curable. In an embodiment, the layers may comprise, from outer to inner, overlacquer/ink/HDPE/adhesive/foil/adhesive/cast polypropylene or overlacquer/ink/PP/adhesive/foil/adhesive/cast polypropylene. In an embodiment, the outer surface of the overlacquer may be electron-beam cured to add heat resistance to the material.

In an embodiment, innermost layer may be heat sealed to the container edge, rim, or flange. As used herein, the term “heat seal” refers to the union of two materials by bringing the materials into contact, or at least close proximity, with one another and then applying sufficient heat and, optionally, pressure to a predetermined area (or areas) of the materials to cause the contacting surfaces of the materials in the predetermined area to become molten and intermix with one another, thereby forming a bond between the two materials in the predetermined area when the heat and, optionally, pressure are removed therefrom and the area is allowed to cool. In accordance with an embodiment of the present invention, a heat seal preferably creates a hermetic seal, i.e., a barrier to the outside atmosphere.

Any suitable method of making a HDPE or PP film having the layers disclosed herein can be used to make a film lidding in accordance with the presently disclosed subject matter. Thus, suitable methods can include (but are not limited to) tubular cast coextrusion, tubular blown coextrusion, flat cast extrusion, coextrusion, extrusion coating, lamination, slot die extrusion, and by other such techniques well known in the art. In an embodiment, a blown film process may be used to form the HDPE or PP film layer. In an embodiment, the HDPE or PP film layer may be machine direction oriented (MDO). In another embodiment, the HDPE or PP film layer may be produced by the cast tentered process.

The adhered film layers may be cut into a lid shape in certain embodiments. The lid may be applied to and/or sealed to a container or may be sterilized separately from the container, in various embodiments. In an embodiment, the lidding film (cut or uncut) is sterilized prior to being sealed to a container. In an embodiment, the container comprises a portion control container (e.g., a container for puddings, sauces, yogurts, etc.) and the lidding film comprises “cut and stack” lidding. In other embodiments, the lidding film may be roll-fed lidding that is trimmed in place after sealing to portion control cups.

Any aseptic sterilization process known in the art may be utilized in the invention. Commercially available aseptic equipment systems may include a tank for hydrogen peroxide, a drying chamber, a container/package section, and a unit which supplies and circulates hydrogen peroxide and controls temperature, air pressure etc. In an embodiment, the lidding film of the invention may be sterilized by hydrogen peroxide set at a temperature of between about 50° C. and about 75° C. In a particular embodiment, the hydrogen peroxide may have a temperature of about 65° C. In an embodiment, the present invention uses hydrogen peroxide with a concentration of approximately 35% or greater. In another embodiment, the present invention uses hydrogen peroxide with a concentration of between approximately 20% to 50%. In an embodiment, liquid hydrogen peroxide immersion, atomized hydrogen peroxide, vaporized hydrogen peroxide, heated sprayed or misted hydrogen peroxide or any other form of hydrogen peroxide may be utilized for sterilization.

Afterward, hot sterile air at a temperature of between about 60° C. and about 80° C. may be used to dry the lidding film and to remove hydrogen peroxide from the film. Temperature and flow level for the hydrogen peroxide may be controlled by steam to raise the temperature, and water may be supplied for cooling.

In an embodiment, the film material will survive in a heated peroxide bath as described herein for up to six hours and, in some case, in excess of thirteen hours, with no distortion, ink smearing, or delamination. In contrast, a typical PET film lidding cannot survive more than about 2 to 4 hours in a heated peroxide bath without becoming distorted, smeared or delaminated.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A method for providing a film lidding structure which can survive aseptic processing, the method comprising:
providing at least one high-density polyethylene (HDPE) film layer;
laminating the at least one HDPE film layer to at least one base layer to form a lidding;
adhering the at least one base layer to the rim of a container to form a lidded container; and
sterilizing the lidded container using high concentration hydrogen peroxide at elevated temperature.

2. The method of claim 1 wherein the at least one HDPE film layer is reverse printed.

3. The method of claim 1 wherein the at least one HDPE film layer comprises an outer surface that is printed and an overlacquer is applied adjacent the printed material.

4. The method of claim 1 wherein the at least one base layer comprises cast polypropylene.

5. The method of claim 1 wherein the film lidding structure comprises the following layers, in order: HDPE, ink, adhesive, foil, adhesive, and cast polypropylene.

6. The method of claim 1 wherein the at least one HDPE film is produced via a blown film process.

7. The method of claim 6 wherein the at least one HDPE film is machine direction oriented.

8. The method of claim 1 wherein the at least one HDPE film is produced via a cast tentered process.

9. The method of claim 1, wherein the concentration of the hydrogen peroxide is between about 20% and about 50%.

10. The method of claim 1, wherein the concentration of the hydrogen peroxide is at least 35%.

11. The method of claim 1, wherein the elevated temperature comprises between about 50° C. and about 75° C.

12. A method for providing a film lidding structure which can survive aseptic processing, the method comprising:
providing at least one high-density polyethylene (HDPE) film layer;
laminating the HDPE film layer to at least one base layer to form a lidding;
sterilizing the lidded container using hydrogen peroxide at a concentration of between about 20% and about 50% and at a temperature of between about 50° C. and about 75° C.; and
adhering the sterilized lidding structure to the rim of a container to form a lidded container.

13. The method of claim 12 wherein the at least one base layer is adhered to the rim of the container.

14. The method of claim 12 wherein the at least one HDPE film layer is reverse printed.

15. The method of claim 12 wherein the at least one HDPE film layer comprises an outer surface that is printed and an overlacquer is applied adjacent the printed material.

16. The method of claim 12 wherein the film lidding structure comprises, in order, HDPE, ink, adhesive, foil, adhesive, and cast polypropylene.

17. The method of claim 12 wherein the at least one HDPE film is produced via a blown film process.

18. The method of claim 17 wherein the at least one HDPE film is machine direction oriented.

19. The method of claim 12 wherein the at least one HDPE film is produced via a cast tentered process.

20. The method of claim 12, wherein the concentration of the hydrogen peroxide is at least 35%.

21. The method of claim 12, wherein the temperature is about 65° C.

22. A method for providing a film lidding structure which can survive aseptic processing, the method comprising:
providing at least one polypropylene film layer;
laminating the at least one polypropylene film layer to at least one base layer to form a lidding;
adhering the at least one base layer to the rim of a container to form a lidded container; and
sterilizing the lidded container using high concentration hydrogen peroxide at elevated temperature.

23. The method of claim 22 wherein the at least one polypropylene film layer is reverse printed.

24. The method of claim 22 wherein the at least one polypropylene film layer comprises an outer surface that is printed and an overlacquer is applied adjacent the printed material.
